(19) United States
(12) Patent Application Publication
(10) Pub. No.: US 2008/0294233 A1
(43) Pub. Date: Nov. 27, 2008

(54) Proximal protection balloon catheter method and device

(76) Inventors: S. Cezar Staniloae, New York, NY (US); Peter G. Kayafas, New York, NY (US); Jerome Priest, North GrosvenorDale, CT (US)

Correspondence Address: PAUL A. FATTIBENE;FATTIBENE & FATTIBENE, 2480 POST ROAD, SOUTHPORT, CT, 06890, US

(21) Appl. No.: 12/148,852

(22) Filed: Apr. 23, 2008

Related U.S. Application Data
(60) Provisional application No. 60/913,953, filed on Apr. 25, 2007.

(51) Int. Cl. A61F 2/84

(52) U.S. Cl. 623/1.11

(57) ABSTRACT

Multiple catheters are used to improve the positioning of devices for the treatment of stenosis in carotid arteries. A first catheter is introduced through the right common femoral artery and used to position a first balloon occluder within the external carotid artery. A second catheter is introduced through the left common femoral artery and used to position a balloon occluder catheter within the common carotid artery after removal of the first catheter. A guide wire is used to guide a self-expanding stent through the balloon occluder catheter adjacent a narrowing or stenosis within the carotid artery for treatment. The utilization of multiple catheters makes possible the positioning of devices used to treat stenosis easier and in locations that have been difficult to treat.

### REFERENCE TO RELATED APPLICATION

This application claims the benefit of U.S. Provisional Patent Application No. 60/913,953, filed Apr. 25, 2007.

### FIELD OF THE INVENTION

The present invention relates generally to the treatment of stenosis of the carotid artery, and more particularly to a method for improved access and treatment of stenosis near the juncture or intersection of the internal and external carotid arteries.

### BACKGROUND OF THE INVENTION

Over time the carotid artery may narrow or develop stenosis. This may be caused by the buildup of plaque that restricts blood flow. Occasionally relatively invasive surgical procedures, such as endarterectomy, may be necessary to remove a blockage or narrowing and increase blood flow. Recently procedures have been developed to avoid the relatively invasive surgical procedure with the use of angioplasty methods. In the angioplasty methods a catheter and guide wire are used to position a balloon or occluder to permit treatment of the stenosis and widening of the carotid artery. While such angioplasty methods have been used to greatly reduce the recovery time of prior surgical techniques, such procedures are not without risk or difficulty. When introducing a catheter or guide wire into the carotid artery there is a risk of dislodging particles or emboli that could travel causing a stroke. The risk for stroke during such procedures may be substantial. This is especially a problem when relatively large devices or apparatus are employed in the angioplasty methods utilized to treat the stenosis.

One such device used for the treatment of carotid artery disease is disclosed in U.S. Pat. No. 6,905,490 issuing on Jun. 14, 2005 to Parodi and entitled “Apparatus and Method for Reducing Embolization During Treatment of Carotid Artery Disease”, which is herein incorporated by reference. Therein disclosed is an apparatus that aids in removing emboli or particles that may be dislodged during the angioplasty-type procedure. A relatively large catheter is used that contains an angioplasty catheter as well as an inflatable balloon utilized for occluding or blocking the external carotid artery. This relatively large catheter containing multiple devices is often relatively difficult to position within the carotid artery and is also more prone to dislodging particles or emboli increasing the risk of stroke. Additionally, the use of a single, relatively large catheter containing multiple devices often makes it more difficult to treat stenosis at or near the intersection of the internal and external carotid arteries. Therefore there is a need to provide a method of treating stenosis in carotid arteries that may be difficult to access or that are in locations that are difficult to treat using existing devices and methods.

### SUMMARY OF THE INVENTION

The present invention relates to a method and kit for treating stenosis in a carotid artery. A first catheter is introduced adjacent to a carotid artery for positioning a balloon occluder within the external carotid artery, removing the first catheter, introducing a second catheter adjacent to a carotid artery for position a balloon occluder catheter within the central carotid artery, removing the second catheter, and treating the stenosis with a stent or other angioplasty procedures through the relatively small balloon occluder catheter. The use of multiple catheters permits the use of smaller catheters that are more easily positioned for the introduction of other devices to treat the stenosis such as balloon occluders, angioplasty balloons, stents, or other similar devices.

Accordingly, it is an object of the present invention to improve the access and treatment of stenosis or blockage of the carotid arteries.

It is a further object of the present invention to reduce the risk of stroke.

It is an advantage of the present invention that the placement of devices used for the treatment of stenosis in a carotid artery is made easier.

It is a further advantage of the present invention that some carotid artery locations that are difficult to access become more easily accessible.

It is yet a further advantage of the present invention that the placement near the intersection of the internal and external carotid arteries is made easier.

It is a feature of the present invention that multiple catheters are used in sequence to position devices for treatment of stenosis in the carotid arteries.

It is another feature of the present invention that a first catheter may be introduced in the right femoral artery and that a second catheter may be introduced in the left femoral artery.

These and other objects, advantages, and features will become more readily apparent in view of the following more detailed description.

### BRIEF DESCRIPTION OF THE DRAWINGS

FIG. 1A-G schematically illustrates the method steps of the present invention.

FIG. 2 is a block diagram illustrating the method steps of the present invention.

FIG. 3 schematically illustrates a balloon occluder cathode used in the present invention.

FIG. 4 schematically illustrates a kit used in the treatment of stenosis in a carotid artery according to the present invention.

### DETAILED DESCRIPTION OF THE PREFERRED EMBODIMENTS

The present invention provides an improved method for the treatment of stenosis in the carotid arteries of a patient. The present invention uses multiples catheters to aid in introducing and positioning devices used in the treatment of stenosis or blockage of the carotid arteries.

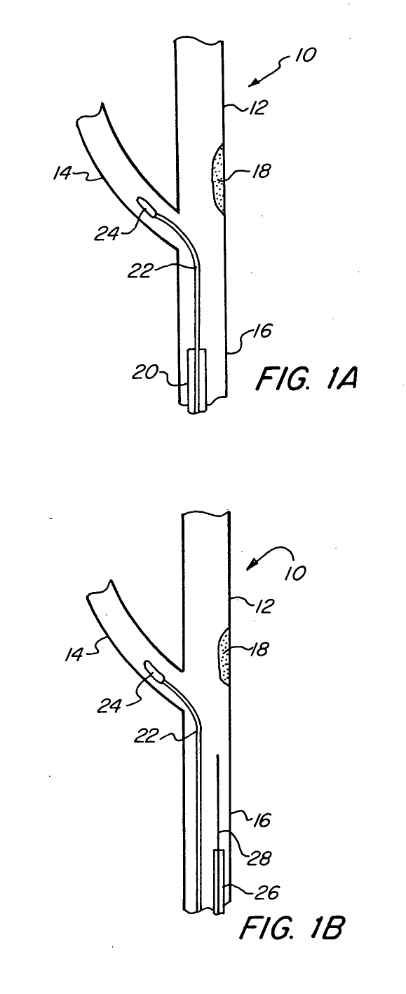

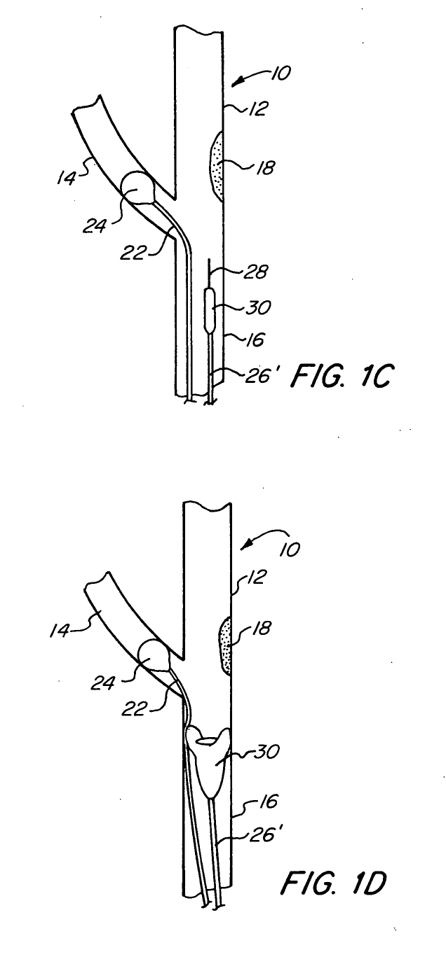

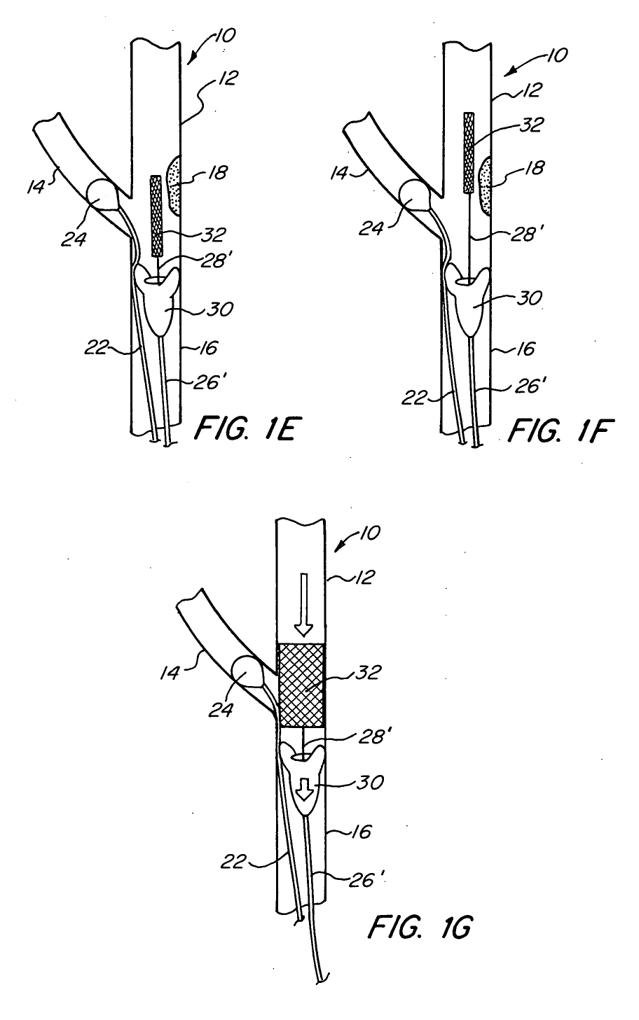

FIGS. 1A-G illustrates the method steps of the present invention used in the treatment of stenosis.

FIG. 1A schematically illustrates carotid arteries and the introduction of a balloon occluder. The method of the present invention begins with the positioning of a number 7 French introducer sheath in the right common femoral artery of a patient and the placement of a number 5 French introducer sheath in a left common femoral artery of a patient. A number 5 French multi-purpose catheter may be introduced through the left common femoral artery and introducer sheath and into the common carotid artery 16. A French is a unit of measure equal to one-third millimeter used in measuring the outside diameter of a tubular instrument inserted into a bodily cavity.

A guide wire 22, which preferably has a diameter of 0.014 inches, is advanced through the catheter 20 into the external carotid artery 14. The catheter 20 may then be removed. A balloon occluder 24 is advanced along the guide wire 22 into position in the external carotid artery 14. A narrowing of the artery or stenosis 18 is illustrated near the intersection of the internal carotid artery 12, the external carotid artery 14, and the common carotid artery 16.

FIG. 1B illustrates the introduction of a second catheter 26, after the removal of the first catheter 20 illustrated in FIG. 1. The second catheter 26 may be a number 5 French multi-purpose catheter that is introduced through the introducer sheath in the right femoral artery. A guide wire 28, which may preferably have a diameter of 0.035 inches, is advanced through the second catheter 26. The second catheter 26 may then be removed.

FIG. 1C illustrates the positioning of an balloon occluder catheter having a balloon occluder 30 and a catheter 26′ advanced over the guide wire 28. Preferably, the balloon occluder catheter has as size of 7 French. The guide wire 28 may then be removed. The balloon occluder 24 positioned within the external carotid artery 14 may then be inflated to stop blood flow within the external carotid artery 14.

FIG. 1D illustrates the inflating of the balloon occluder 30 within the common carotid artery. Accordingly, there should be a back flow of blood away from the stenosis 18 and directed toward the balloon occluder 30.

FIG. 1E illustrates the insertion of guide-wire 28′, which preferably may a diameter of 0.014 inches, through the catheter 26′ having a self-expandable stent 32 thereon.

FIG. 1F illustrates the positioning of the self-expandable stent 32 adjacent the stenosis 18 along guide wire 28. The proximal end of the stent 32, which may or may not end in the common carotid artery. The self-expanding stent 32 may be post-dilated with an adequately sized dilation balloon. A back flush of approximately 50 cc of blood may be performed through the catheter 26′.

FIG. 1G illustrates the self-expanding stent 32 in position relieving the narrowing or stenosis within the carotid artery. The balloon occluder 24 within the external carotid artery 14 may now be deflated as well as the balloon occluder 30 within the common carotid artery. A final angiogram may be performed to confirm that the stent is positioned properly and that there are no additional complications. The balloon occluder 24 within the external carotid artery may be removed along the guide wire 22 through the left common femoral artery. The stent 32, being somewhat compliant, will permit the deflated balloon occluder 24 to pass. However, some caution must be exercised when removing the deflated balloon occluder 24 so as not to move the self-expanding stent 32. The deflated balloon occluder 30 within the common carotid artery may also be removed along guide wire 28′ through the right common femoral artery.

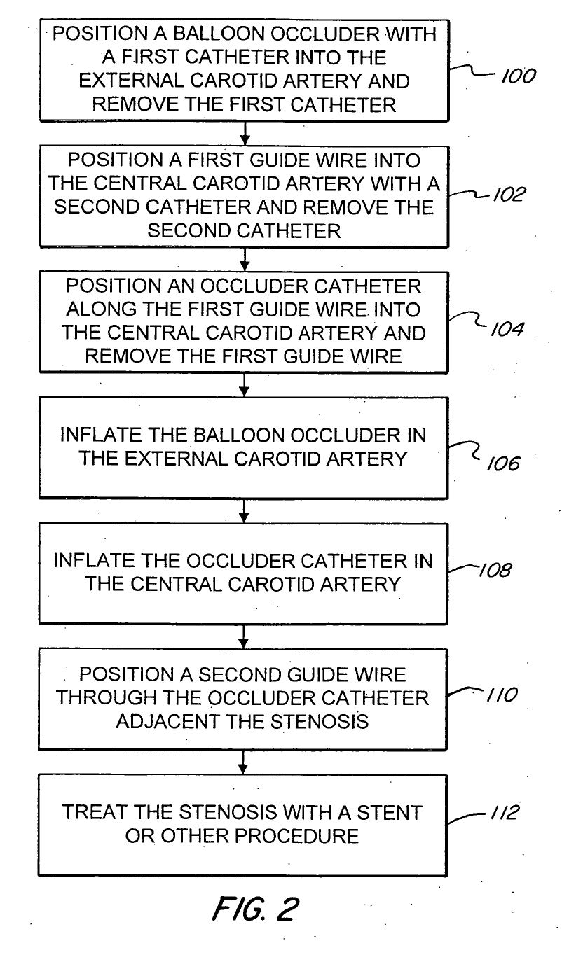

FIG. 2 is a block diagram illustrating method steps of the present invention. Block 100 represents the method step of positioning a balloon occluder with a first catheter into the external carotid artery, and then removing the first catheter. Block 102 represents the method step of positioning a first guide wire into the central carotid artery with a second catheter, and then removing the second catheter. Block 104 represents the method step of positioning a balloon occluder catheter along the first guide wire into the central carotid artery, and then removing the first guide wire. Block 106 represents the method step of inflating the balloon occluder in the external carotid artery. Block 108 represents the method step of inflating the balloon occluder catheter in the central carotid artery. Block 110 represents the method step of positioning a second guide wire through the balloon occluder catheter adjacent the stenosis. Block 112 represents the method step of treating the stenosis with a stent or other angioplasty type procedure to improve blood flow. After treating the stenosis the balloon occluder and the balloon occluder catheter may be deflated and removed.

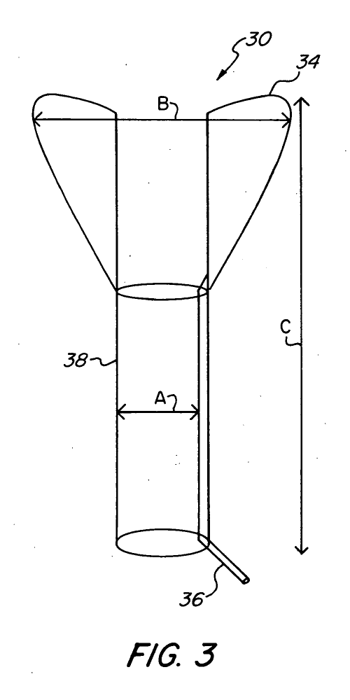

FIG. 3 schematically illustrates a balloon occluder catheter 30 that may be used in practicing the present invention. The balloon occluder catheter 30 comprises a balloon occluder 34, a generally cylindrical catheter portion 38, and a balloon inflator 36. Generally, the diameter A of the catheter is approximately 2.06 mm or 0.81 inches. The approximate diameter B range of the balloon inflator 26 is between 7 mm to 12 mm. The entire length C of the balloon occluder catheter 30 may be approximately 90 cm. The balloon occluder catheter 30 may have a concave shape that reduces the risk of plaque accumulation in the shadow areas between the balloon and the vessel or artery wall.

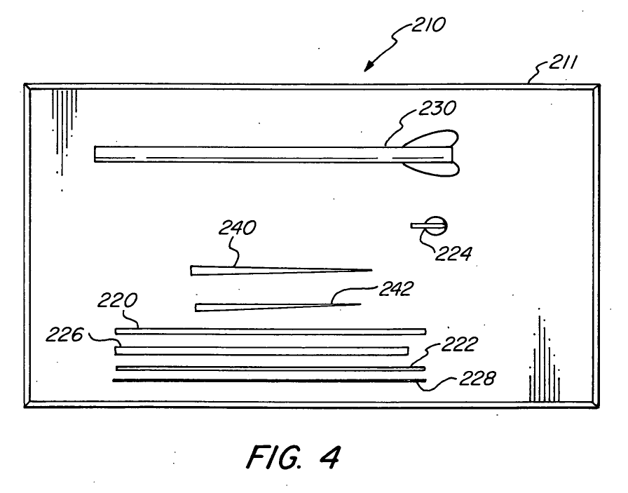

FIG. 4 schematically illustrates a kit for treating stenosis of the carotid artery. The kit 210 comprises a package 211 containing a balloon occluder catheter 230, a balloon occluder 224, a first introducer sheath 240, preferably having a size of 7 French, a second introducer sheath 242, preferably having a size of 5 French, a first and second multipurpose catheter 220 and 226, preferably having a size of 5 French, a first guide wire 222, preferably having a diameter of 0.014 inches, and a second guide wire 228, preferably having a diameter of 0.035 inches. The kit of the present invention greatly facilitates the practicing of the method steps of an embodiment of the present invention.

The present invention by using multiple catheters to introduce devices to treat stenosis of the carotid artery makes possible the use of much smaller devices resulting in less trauma and the ability to position the devices more easily and with less risk of emboli and possible stroke.

While the present invention has been illustrated and described with respect to a preferred embodiment, it will be obvious to those skilled in the art that various modifications may be made without departing from the spirit and scope of this invention.

What is claimed is:

1. A method of treating a stenosis in a carotid artery comprising the steps of:
introducing a first catheter into an artery for positioning a first guide wire and a balloon occluder within an external carotid artery;
removing the first catheter;
introducing a second catheter into an artery for positioning a second guide wire and balloon occluder catheter within a common carotid artery;
removing the second catheter;
inflating the balloon occluder and the balloon occluder catheter, whereby blood flow is caused to be directed away from the stenosis and towards the balloon occluder catheter; and
treating the stenosis improving blood flow,
whereby devices for treating the stenosis are easily placed within the arteries.

2. A method of treating a stenosis as in claim 1 wherein:
said step of treating the stenosis comprises positioning a self expanding stent adjacent the stenosis.

3. A method of treating a stenosis as in claim 1 wherein:
said step introducing a first catheter comprises the step of introducing the first catheter through a common femoral artery; and
said step introducing a second catheter comprises the step of introducing the second catheter through another common femoral artery.

4. A method of treating a stenosis as in claim 1 wherein:
the balloon occluder catheter has a size of approximately 7 French.

5. A method of treating stenosis in a carotid artery comprising the steps of:
inserting an first introducer sheath into a right common femoral artery;
inserting an first introducer sheath into a left common femoral artery;
inserting a first catheter through the first introducer sheath and the right common femoral artery into a common carotid artery;
advancing a first guide wire through the first catheter into an external carotid artery;
removing the first catheter;
advancing a balloon occluder over the first guide wire into the external carotid artery;
inserting a second catheter through the second introducer sheath into a common carotid artery;
advancing a second guide wire through the second catheter into the common carotid artery;
removing the second catheter;
advancing a balloon occluder catheter over the second guide wire into the common carotid artery;
removing the second guide wire;
inflating the balloon occluder within the external carotid artery;
inflating the balloon occluder catheter within the common carotid artery;
advancing a third guide wire through the balloon occluder catheter into a internal carotid artery adjacent the stenosis;
treating the stenosis;
deflating the balloon occluder within the external carotid artery;
deflating the balloon occluder catheter within the common carotid artery;
remove the balloon occluder; and
remove the balloon occluder catheter,
whereby the stenosis is treated with reduced risk of emboli and possible stroke.

6. A method of treating stenosis as in a carotid artery as in claim 5 wherein:
said step of treating the stenosis comprises positioning a self expanding stent adjacent the stenosis.

7. A kit for treating stenosis of a carotid artery comprising:
a package;
a balloon occluder catheter placed within said package;
a first and second introducer sheaths placed within said package;
a first and second catheter placed within said package; and
a first and second guide wire placed within said package.
